(19) United States
(12) Patent Application Publication
(10) Pub. No.: US 2024/0215566 A1
(43) Pub. Date: Jul. 4, 2024

(54) Device and Method for Handling, Packaging, Storing, Transporting, Applying and Releasing Biological Pest Control Agents and Other Substances

(71) Applicants: Ricardo Antonio DE OLIVEIRA MACHADO, Sao Paulo (BR); Nicholas RODWELL MATIAS, Sao Paulo (BR)

(72) Inventors: Ricardo Antonio DE OLIVEIRA MACHADO, Sao Paulo (BR); Nicholas RODWELL MATIAS, Sao Paulo (BR)

(21) Appl. No.: 18/557,985

(22) PCT Filed: Nov. 10, 2022
(86) PCT No.: PCT/BR2021/050211

§ 371 Date: Oct. 30, 2023

(30) Foreign Application Priority Data
May 4, 2021 (BR) 10 2021 0088612-2

(51) Int. Cl. A01M 99/00; A01C 7/08; A01K 1/08; A01K 5/02; A01K 67/033; A01M 9/00

(52) U.S. Cl. CPC A01M 99/00; A01K 1/08; A01C 7/08; A01K 5/02; A01K 67/0332; A01M 9/0007

(57) ABSTRACT

The biological control of agricultural pests is growing rapidly due to the increasing trend towards healthier foodstuffs. The present invention is a response to this and aims to enhance productivity and optimize the entire biological control chain, from operations related to breeding, production, handling, and storage of organisms, as well as transportation, release, and field application. This reduces costs and increases efficiency in the sustainable management of pests, optimizing the quality and viability of biological crop protection products, which include but are not limited to chemical powders, minerals, microbiological agents, insects in the form of eggs, pupae and adults, earthworms, animal feed, seeds, or fruits. These products require specific climatic conditions, and their physical integrity must be maintained, minimizing stress and damage from production to release into the environment. To improve the operation of this system, the concept of using cartridges and refills has been developed.

### BACKGROUND

Biological pest control involves the creation of living organisms (biological control agents) in laboratories (insectaries, or other suitable environment), which are released into the environment to prey on or parasitize pests, reducing infestations.

Currently at the end of production, biological control agents are packaged in bottles, plastic cups, tubes or other packaging for this purpose.

After packaging, the containers are transported to the release site. The release process is carried out manually, where the containers are opened individually, and the organisms are dispersed. There are also cases where the organisms are transferred from their transport packaging in the field to an applicator mechanism, which releases them into the environment. Another technique involves releasing the organisms directly into the field within their transport containers.

These methods of transporting, loading, and releasing biological control agents are inefficient because they involve multiple steps. They also expose workers to the environment, generate waste when disposing containers in the field, and carry a high risk of inducing stress and damage to the transported biological organisms.

The present invention provides equipment and methods to optimize the logistics chain of biopesticides, from production to release into the environment. It minimizes the steps in the process, reduces the handling of biological control agents, and packages them in a way that preserves their physical integrity and post-release viability.

### SUMMARY

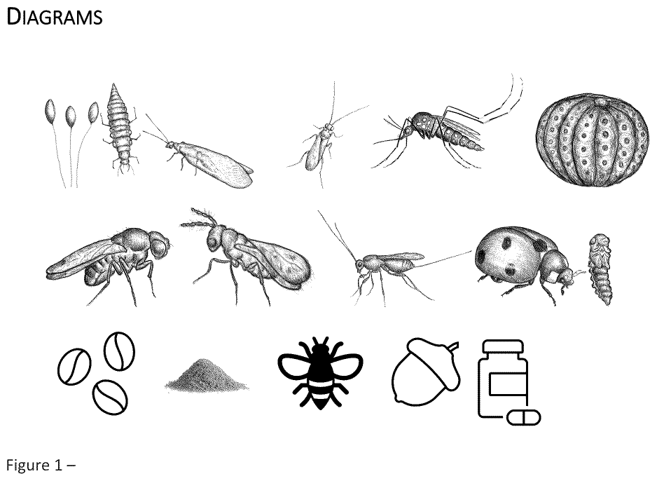

The biological pest control sector in agriculture is rapidly growing. Considering the trend towards healthier foods produced with lower toxicity and less environmental degradation, the demand for this type of pest management is expected to increase in the coming years. FIG. 1 provides examples of some biological control agents and other substances that can be packaged, transported, and applied using the present invention. These substances include, but are not limited to, chemical powders, minerals, microbiological agents, insects in various life stages (eggs, pupae, adults), earthworms, animal feed, seeds, or fruits.

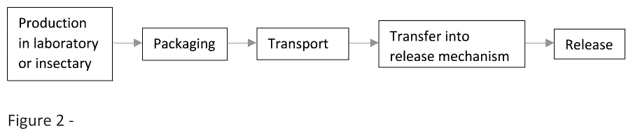

The increasing demand for biological pest control agents underscores the need for new systems and devices for handling, packaging, storing, transporting, and releasing these organisms. Given the nature of these substances, they must be kept under specific climatic conditions, and their physical integrity must be preserved with minimal stress, avoiding damage, from their production in the insectary to the moment of release into the environment. FIG. 2 illustrates the typical cycle of the biopesticide chain.

The proper handling of substances used in biological pest control is undergoing modernization due to the ease introduced by the use of drones or UAVs (Unmanned Aerial Vehicles) currently in existence and under development. For this set up to function effectively, it is crucial to create a new system for handling, packaging, transporting, and releasing substances. This system should help reduce substance wastage during application, increase its effectiveness compared to the current system, and improve productivity to levels capable of economically impacting the business.

The concept of using cartridges and refills with the potential for reusing certain processes and components helps in reducing costs, enhancing efficiencies, and improving the quality of the final product. The biological pest control industry has characteristics that make it conducive to leveraging the effectiveness of the cartridge and refill concept.

The present invention aims to optimize the entire biological control supply chain, from the operations of breeding, production, handling, and storage of organisms to transportation, release, and application in the field, improving overall productivity per workday. This reduces costs, enhances effectiveness, and promotes the use of sustainable pest management, leading to gains in the quality and viability of the biopesticide used.

### DETAILED DESCRIPTION

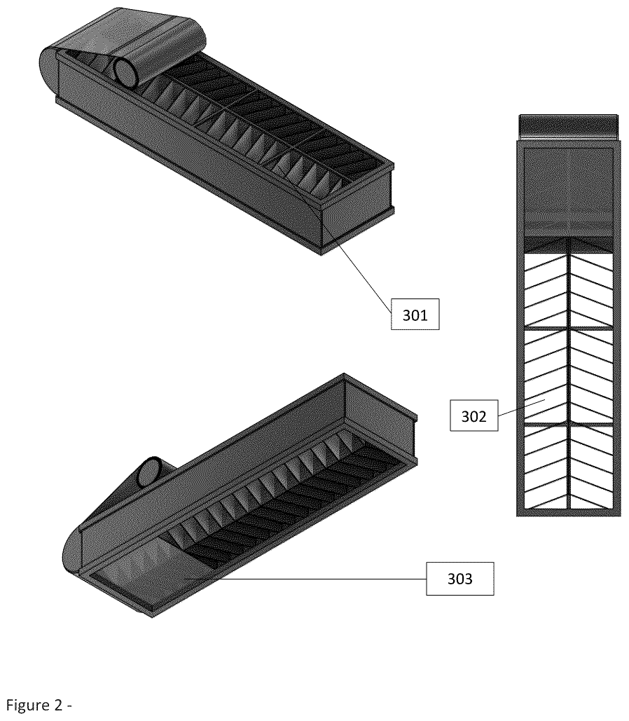

The present invention, as illustrated in FIG. 3, incorporates a structure (FIG. 3, item 301) that defines specific internal volumes (FIG. 3, item 302) with sufficient rigidity to prevent crushing, damage, or harm to the substance carried internally. This structure contains barriers on one or more sides (FIG. 3, item 303) that act to retain the payload substance within the internal volumes of the packaging until the moment of release into the environment. Certain substances can be accommodated in the packaging without the need for barriers. The packaging, now translated into a cartridge with internal and external barriers, can be made from a variety of materials, such as synthetic or natural polymers (such as plastics, bio-plastics, elastomers, alginates, gelatins, among others), textiles (such as natural or synthetic fabrics), cellulose-based materials (such as papers, cardboard, cardstock, papier-mâché), or any other material that provides the necessary conditions for the storage, transportation, and release of the substances.

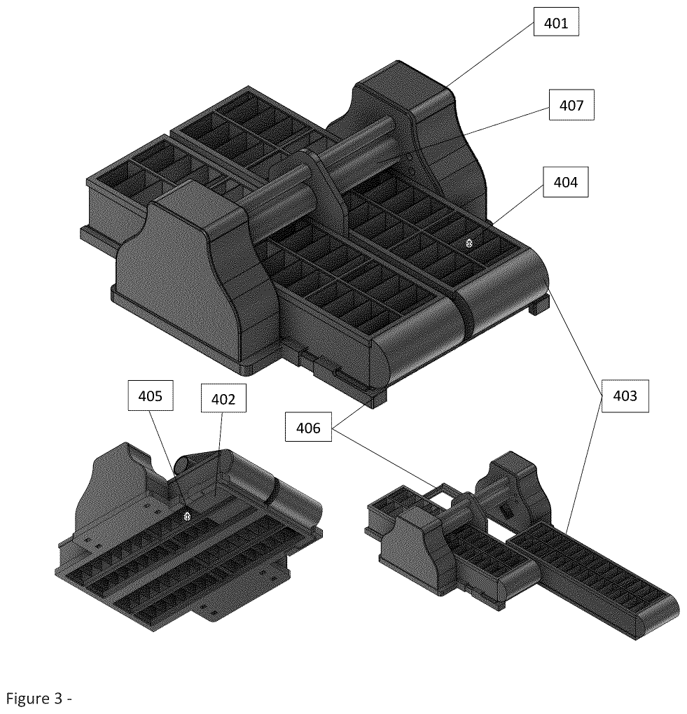

The biological control agents or other substances are loaded into the cartridge. In the case of biological agents, they can be loaded at any stage of their life cycle, and they can even be raised within the internal cavities of the cartridge. The cartridge is prepared at the insectary and transported in a suitable container until the release moment, where it can be fitted into a structured mechanism to receive it, avoiding additional handling (FIG. 4, item 401).

At the release moment, the lower barrier (FIG. 4, item 402) of the cartridge (FIG. 4, item 403) performs a specific action to trigger the controlled release of the payload (FIG. 4, item 404) into the environment, exposing the substances to the force of gravity in a controlled manner (FIG. 4, item 405), which depicts the substance at the moment of release.

In the variant with a retractable barrier (FIG. 4), the lower barrier (FIG. 4 item 402) is fitted into a rail system at the bottom of the cartridge (FIG. 4 item 406). This allows the cartridge to engage with the mechanism defined by the patent application (WO 2021/007632 A1), and the lower barrier has a tab connected to the motor shaft (FIG. 4 item 407), in this case, a rotational shaft. The rotational movement of the shaft, acting as a reel, causes the sliding of the barrier, sequentially exposing the internal cavities of the cartridge, allowing gravity to release and dispense the payload into the environment. This sliding movement can be manually or automatically activated, with the possibility of controlling aspects such as speed, acceleration, and the sequence of movements, among others.

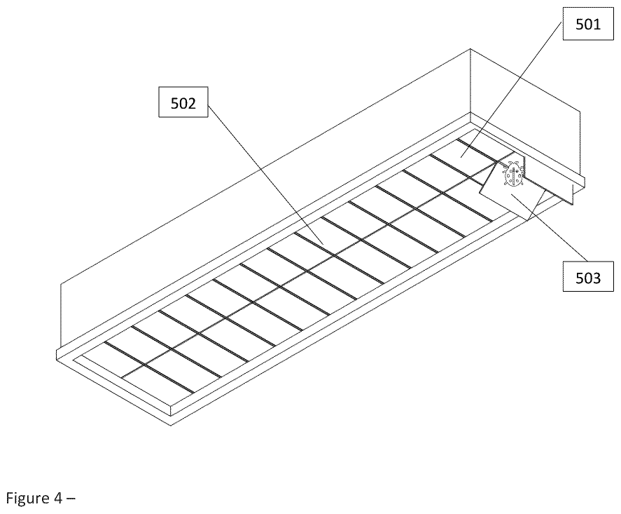

In the variant with a tilting barrier (FIG. 5), the lower barrier (FIG. 5 item 501) is divided in such a way as to cover each internal cavity of the cartridge independently. This can be achieved using a hinge mechanism (FIG. 5 item 502), leveraging mechanical properties of the materials, or even a combination of materials. Each barrier has an actuator element that, once directly or indirectly coupled to the motor shaft, enables the individualized opening of each internal cavity of the cartridge (FIG. 5 item 503).

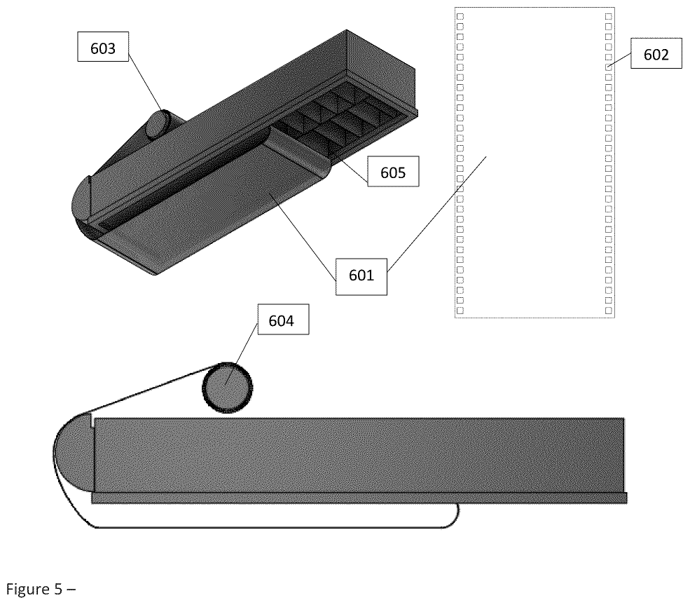

In the variant with a tearable barrier (FIG. 6), the lower barrier (FIG. 6 item 601) can be affixed using adhesive or mechanical coupling on the sides of the cartridge. The material of the lower barrier can be perforated at its sides (FIG. 6 item 602), allowing the center of the barrier to be detachable. The extreme tip of the lower barrier (FIG. 6 item 603) is directly or indirectly coupled to the motor element (FIG. 6 item 604), which, acting as a reel, causes the controlled tearing or detachment of the lower barrier, resulting in the controlled exposure of the internal cavities of the cartridge (FIG. 6 item 605). The detachment or tearing of the barrier can also be done manually.

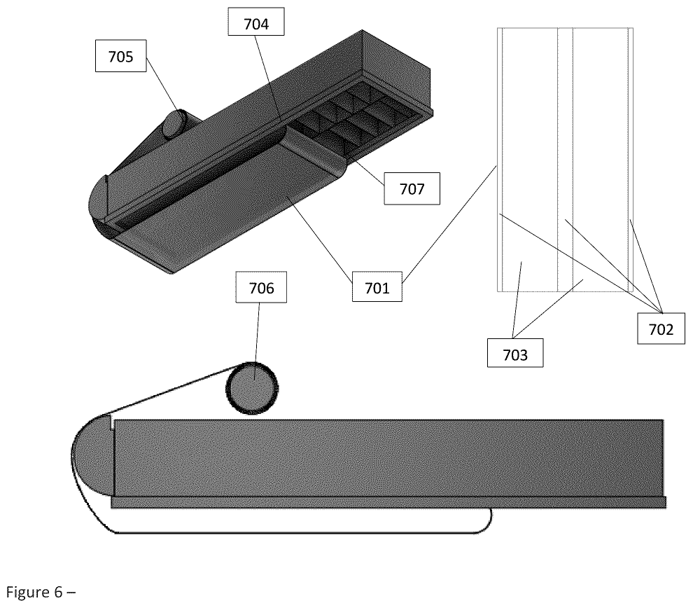

In the variant with an adhesive barrier (FIG. 7), the lower barrier (FIG. 7 item 701) can have its sides or other parts made adhesive (FIG. 7 item 702), and non-adhesive portions (FIG. 7 item 703). This way, the lower barrier can be affixed by adhesive bonding to the sides (FIG. 7 item 704) or other structures of the cartridge. The extreme tip of the barrier (FIG. 7 item 705) can be directly or indirectly coupled to the motor element (FIG. 7 item 706), which, acting as a reel, causes the controlled detachment of the sides of the lower barrier, resulting in the controlled exposure of the internal cavities of the cartridge (FIG. 7 item 707). The detachment of the barrier can also be done manually.

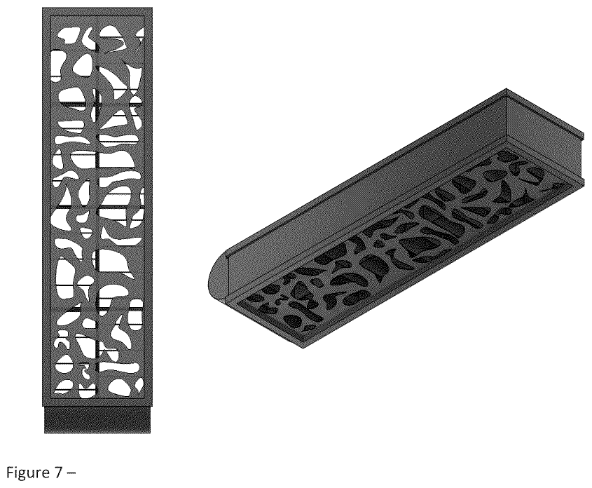

In the variant with a corrosible barrier (FIG. 8), the lower barrier is made of a material that corrodes during the release. It can be made from materials such as paper, gelatin, alginates, or other corrosible substances, and the lower barrier is attached to the cartridge either mechanically or by adhesive. At the time of release, the cartridge is exposed to the environment, which initiates a corrosion process on the lower barrier, leading to the exposure of the internal cavities of the cartridge to the environment.

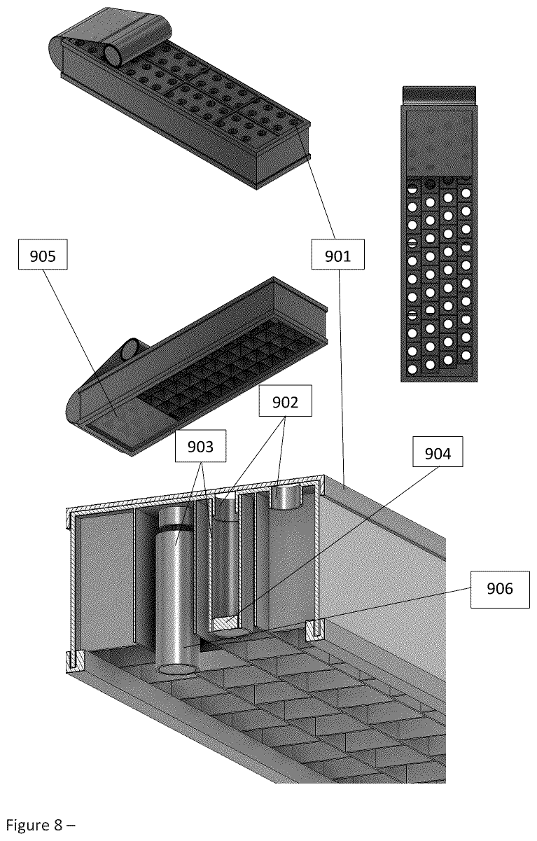

In the variant with vertically enclosed payload (FIG. 9), the upper barrier (FIG. 9 item 901) can incorporate stabilizing elements (FIG. 9 item 902) for the enclosures (FIG. 9 item 903), which serve to maintain the orientation of the packages and to assist in loading insects with food, bait, light, or other attractants directly into the enclosures. These enclosures may or may not have a blocked end (FIG. 9 item 904) to contain the substances. In this modality, the lower barrier (FIG. 9 item 905) can be activated in any way, and the cartridge and enclosure can be designed in any geometric shape for the controlled or random release of the payload (FIG. 9 item 906).

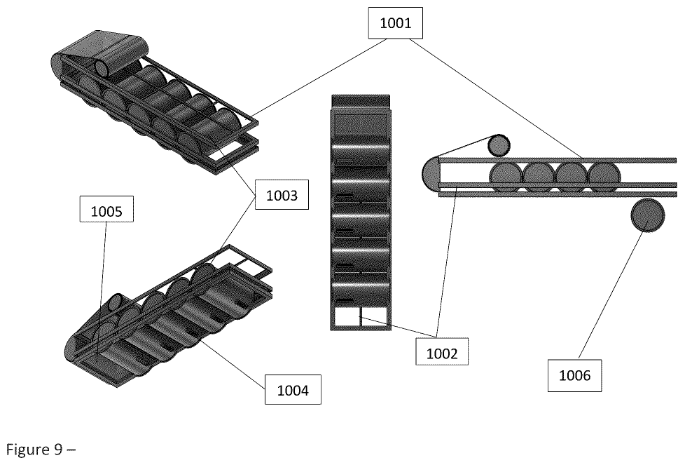

In the variant with horizontally enclosed payload (FIG. 10), the upper barrier (FIG. 10 item 1001) can incorporate stabilizing elements (FIG. 10 item 1002) for the enclosures (FIG. 10 item 1003), which serve to maintain the orientation of the packages and to assist in loading insects with food, bait, light, or other attractants directly into the enclosures. These enclosures may or may not have a blocked end (FIG. 10 item 1004) to contain the substances. In this modality, the lower barrier (FIG. 10 item 1005) can be activated in any way, and the cartridge and enclosure can be designed in any geometric shape for the controlled or random release of the payload (FIG. 10 item 1006).

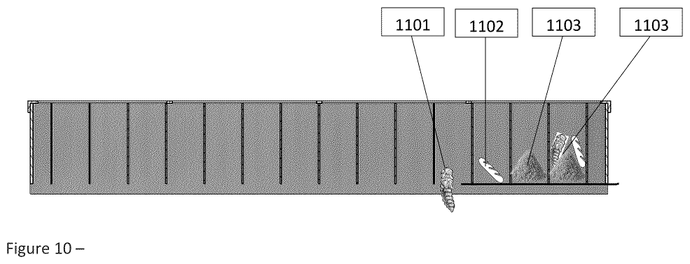

The cartridges can be loaded with insects in various ways, in any viable combination of organisms. In FIG. 11, pupae (FIG. 11 item 1101), masses (FIG. 11 item 1102), and insect eggs (FIG. 11 item 1103) can be manually loaded, either individually or in a formulated manner (FIG. 11 item 1104). Insects applied in the form of adults can be loaded using airflows, mechanically with sedated or knocked down insects, or by employing natural attractants for insects such as light, bait, pheromones, among others.

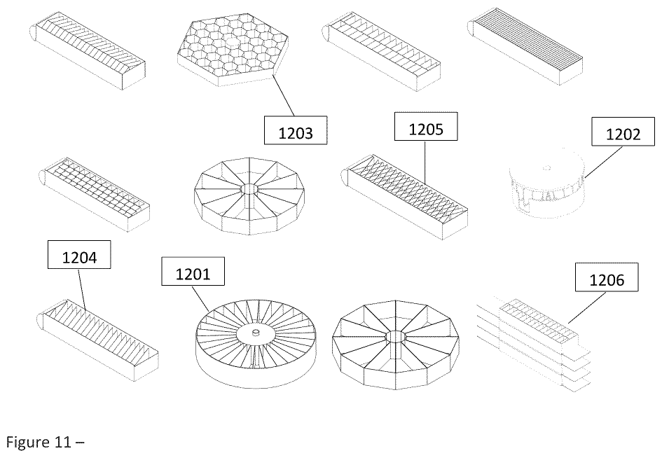

The cartridge structure can be designed in various shapes to accommodate the characteristics of the substance to be released. Having at least one internal cavity, they can be arranged as shown in FIG. 12, in formats such as Vertical Barrel (FIG. 12 item 1201), Radial Barrel (FIG. 12 item 1202), Polygonal (FIG. 12 item 1203), Simple Rectangular (FIG. 12 item 1204), Complex Rectangular (FIG. 12 item 1205), Complex Vertical Rectangular (FIG. 12 item 1206), or in any other suitable configuration.

Vertical Barrel Variant—FIG. 12, item 1201. In this modality the cartridge has a ring shape, the releasing face, lower barrier, is located on the bottom face, and can be removed in order to expose the internal cavities, and release the substance down towards the ground.

Radial Barrel Variant—FIG. 12, item 1202. In this modality, the cartridge has a ring-shaped format, with the release surface located on the exterior of the ring. By removing the external barrier in this configuration, the substances are released radially from the center of the cartridge.

Polygonal Variant—FIG. 12, item 1203. In this modality both the format of the cartridge and the shape of the internal cavities can be of any geometric format for the packaging and release of the substances.

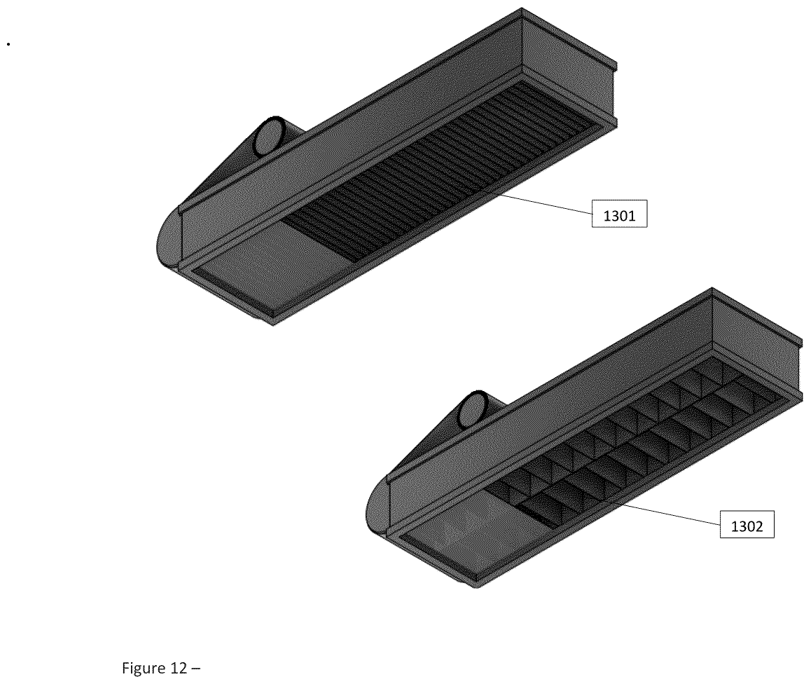

Simple Rectangular—FIG. 13. In this mode, the cartridge contains at least one row of internal compartments (FIG. 13, item 1301). In versions with two or more rows, the rows can be parallel, or they can be offset (FIG. 13 item 1302), allowing greater control over the release of substances.

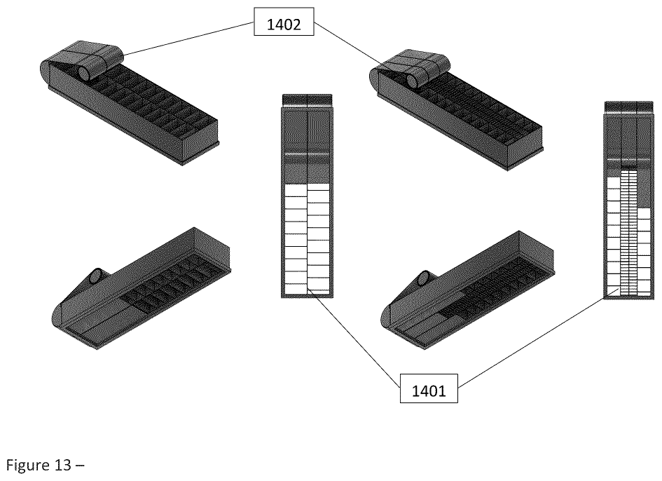

Complex rectangular—FIG. 14. In this modality the cartridge contains at least two rows of internal compartments, and each row can have compartments of different volumes to accommodate different substances (FIG. 14 item 1401). Each row can also be driven independently by one or more motors (FIG. 14, item 1402), allowing more granular control over the released substance.

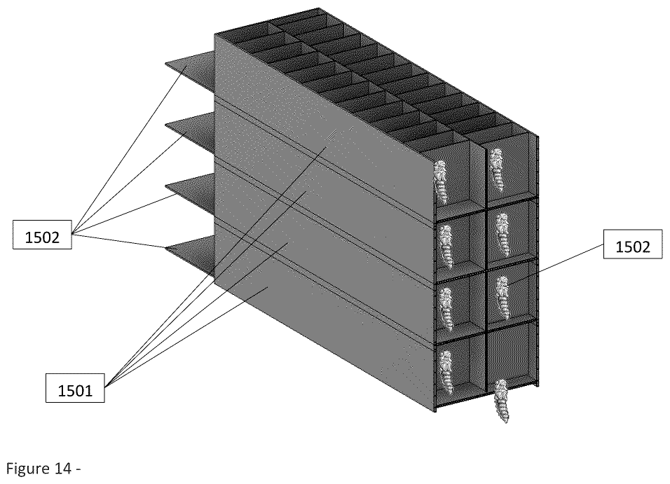

Complex Vertical Rectangular—FIG. 15. In this modality, at least two cartridges (FIG. 15 item 1501) are stacked vertically, and each one has its own lower barrier (FIG. 15 item 1502), which can be activated collectively or individually, allowing for an increase in the onboard payload (FIG. 15 item 1503) and granular control over its release.

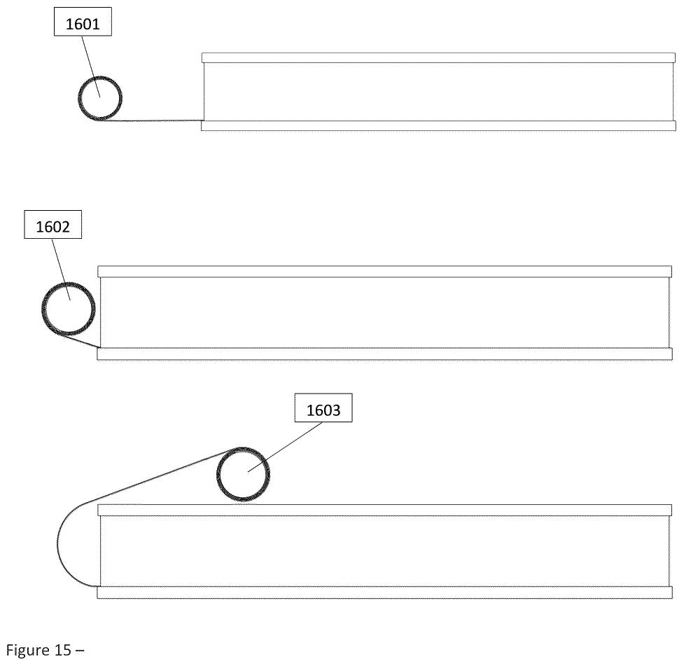

The motor shaft that controls the actions of the lower barriers—FIG. 16, can be located at any position relative to the cartridge, including but not limited to the axis parallel to the movement of the barrier (FIG. 16 item 1601), semi-offset axis (FIG. 16 item 1602), offset axis (FIG. 16 item 1603), or any other suitable configuration.

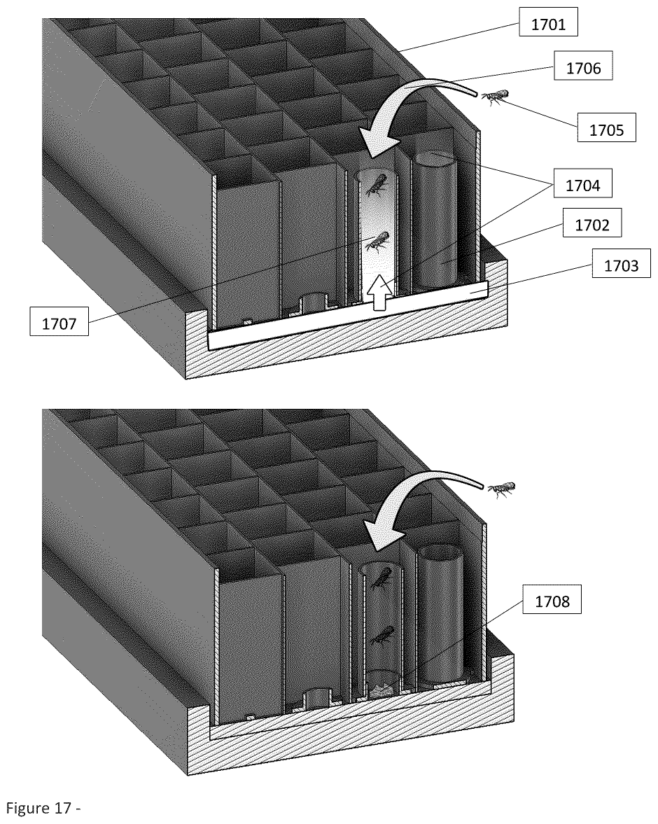

Any current or future variant of the cartridge, as well as any other enclosure, container, or packaging, can utilize light or natural and synthetic attractants to lure biological agents into their interiors for subsequent storage, transport, and release. To illustrate the method, FIG. 17 shows a cartridge (FIG. 17 item 1701) with and without an enclosure (FIG. 17 item 1702). The cartridge is placed near a light table or another light source (FIG. 17 item 1703), which features selective sealing of the light to direct the light radiation only into the internal cavities of the cartridges or enclosures (FIG. 17 item 1704). In this way, the light acts as an attractant to the biological organism (FIG. 17 item 1705), which moves towards it (FIG. 17 item 1706) until it is enclosed within the enclosure (FIG. 17 item 1707), cartridge, or another packaging, where it will be retained until the moment of release. This method can also be used with the substitution of light for another natural or synthetic attractant (FIG. 17 item 1708), including but not limited to pheromones, food baits, odoriferous baits, or other biological agents and substances attractive to biological control agents.

1-4. (canceled)

5. A device for handling, filling, storing, transporting, applying and releasing biological pest control agents and other substances, comprising:
defined internal volumes having internal and external barriers with sufficient rigidity to maintain the physical integrity of the payload, wherein the device is manufactured from synthetic or natural polymers that are textile or cellulosic, and wherein the device is configured to accommodate payloads of biological pest control agents or other substances to agriculture, enveloped or not.

6. The device of claim 5, further comprising a payload of substances including biological agents ready for release, which are inserted mechanically, manually, by airflow, or by natural attractants including light, baits, or pheromones, being able to allow the evolution of stages of the life cycle of biological agents within the cartridge cavities, and the device configured to be in formulation for release in a different life stage from that in which it was loaded, and also a payload of other beneficial substances, including chemical powders, minerals, microbiological agents, insects in eggs, pupae, larvae, adults, earthworms, animal feed, capsules, granules, pheromones, seeds or fruits.

7. The device of claim 5, wherein the device is a Vertical Barrel format whose releasing face is located at the bottom, and is removable to expose the internal cavities and allow the controlled release of the substances.

8. The device of claim 5, wherein the device is a Radial Barrel format with the external release face being removable, allowing the radial release of substances from the center of the device.

9. The device of claim 5, wherein the device is a Polygonal format where the cartridge and the internal cavities are geometrically shape as quadrangular, rectangular, triangular, circular, hexagonal, arranged in a line, or parallel lines for packaging and release of substances.

10. The device of claim 5, wherein the device is a Simple Rectangular format containing at least one row of internal compartments, and is configured with two or more parallel or offset rows.

11. The device of claim 5, wherein the device is a Complex Rectangular format containing at least two rows of internal compartments, each row can having compartments of different volumes and geometries to accommodate various substances that can be triggered and released independently by the differential operation of the lower barriers.

12. The device of claim 5, wherein the device is a Complex Vertical Rectangular format where at least two cartridges are stacked vertically so that the internal cavities are aligned, allowing independent operation of the lower barriers of each cartridge.

13. The device of claim 5, further comprising a lower barrier where at the time of release the lower barrier of the cartridge performs an action to promote the controlled release of the payload, exposing the substances in a controlled manner.

14. The device of claim 13, wherein by having a retractable barrier, where the lower barrier is fitted into rails at the bottom of the cartridge, with its extremity connected to a rotary motion motor shaft, which, acting as a reel slides the barrier and sequentially exposes the internal cavities of the cartridge, allowing the controlled release of substances.

15. The device of claim 13, wherein by containing a tilting barrier, where the lower barrier is divided to cover each internal cavity of the cartridge independently, allowing individualized opening of each cavity.

16. The device of claim 13, further comprising tearable barriers at the lower barrier, with perforated or serrated sides is fixed by adhesive or mechanical means on the sides of the cartridge, allowing the center to be torn by a driving force.

17. The device of claim 13, further comprising adhesive barriers at the sides or central parts of the lower barrier, with the tip of the lower barrier connected directly or indirectly to the motor element that undoes the fixation of the sides of the lower barrier, allowing controlled release of substances.

18. The device of claim 13, wherein having a corrosive barrier at the lower barrier is made of a material that corrodes during release, the cartridge is exposed to the environment, initiating a corrosion process on the lower barrier, exposing the internal cavities and releasing substances.
